(19) United States
(12) Patent Application Publication
(10) Pub. No.: US 2020/0335181 A1
(43) Pub. Date: Oct. 22, 2020

(54) DIGITAL REACTIONWARE

(71) Applicant: THE UNIVERSITY COURT OF THE UNIVERSITY OF GLASGOW, Glasgow Strathclyde (GB)

(72) Inventor: Leroy CRONIN, Glasgow Strathclyde (GB)

(21) Appl. No.: 16/960,707

(22) PCT Filed: Jan. 9, 2019
(86) PCT No.: PCT/EP2019/050439

§ 371 Date: Jul. 8, 2020

(30) Foreign Application Priority Data
Jan. 9, 2018 (GB) 1800299.8

(51) Int. Cl. G16C 20/10; B01L 3/00; B33Y 10/00; B33Y 30/00

(52) U.S. Cl. CPC G16C 20/10; B01L 3/52; B01L 2200/16; B33Y 30/00; B33Y 10/00

(57) ABSTRACT

The invention provides a method for digitising a method of synthesis. The method includes the steps of identifying a method of synthesis for a target product; (ii) establishing a process sequence for that method, which process sequence is a collection of chemical and/or physical steps within the method of synthesis; and subsequently (iii) translating the process sequence to a digital model of the method of synthesis, which digital model comprises a digital description of the chemical and/or physical steps within the method of synthesis.

### RELATED APPLICATION

The present application claims the benefit of and priority to GB 1800299.8, filed on 9 Jan. 2018 (09.01.2018), the contents of which are hereby incorporated by reference in their entirety.

### FIELD OF THE INVENTION

The present invention provides methods for the preparation of a reactionware for performing a multistep synthesis, methods of organic synthesis using the reactionware, such as on demand synthesis, as well as the reactionware itself. Also provided is a method of digitising a synthesis and preparing a digital model of the reactionware for the synthesis. Also provided are interchangeable and adaptable digital and chemical reaction modules for use in organic synthesis.

Background

The manufacture of active agents, such as active pharmaceutical ingredients (APIs), is vital for modern healthcare, yet critical drugs are regularly manufactured for a finite period in a limited number of specialist sites. The manufacture of chemical products, whether they be bulk, fine or specialty chemicals, is currently based on a model whereby a central plant is exclusively designed for the manufacture of the product, or range of products, sold by that particular company (see Roberge et al.).

This model holds whether the manufacturer be a large pharmaceutical company or, as is increasingly the case, a contract research organization (CRO) operating large chemical manufacturing plants to order from the pharmaceutical industry. This process leads to safety issues around both the storage and transport of reagents, intermediates and products, as well as the issues inherent in the large scale manufacture of chemicals (see Crowl et al.).

In addition, these large scale plants are often at the mercy of complicated and global supply chains of raw materials, the failure of which at any point will reduce or halt the capacity of the plant to produce materials and deliver them effectively. Also, when a given complex intermediate or active agent, such as an API, goes out of production, the plants are typically repurposed and the manufacturing capacity specific for those intermediates or active agents is lost.

The reestablishment of the process would require, in the best case, substantial capital investment to reconfigure a chemical plant for its synthesis, as well as the time needed for the repurposing.

Some target compounds, such as radioactive tracers, degradable dyes for biological labelling, and other short life compounds may be too unstable or short-lived to be made on an industrial scale, and methods are required for the on-demand generation of such compounds at an appropriate level of scale and purity. Where the preparation of such compounds is complex, it is clearly desirable to provide processes that will allow an unskilled user to prepare those compounds reliably and without great difficulty.

WO 2013/121230 describes method for performing chemical reactions in bespoke reactionware. A 3D printer is used to prepare a reactionware of programmed dimensions and structure. The reactions may be performed in the reactionware as desired, and advantageously the 3D printer may be used to deliver reagents, solvent and catalysts to the reactionware, as wells as being used to prepare the reactionware itself.

Whilst WO 2013/121230 highlights the usefulness of 3D printing techniques to prepare reactionware, WO 2013/121230 does not explicitly describe the translation of multistage synthetic procedures from a traditional synthetic route into an on-demand printed architecture.

There is need therefore to develop techniques to allow for reproducible preparation of desirable active agents according to established and reliable protocols, and to do so for complex, multistage synthetic procedures. This can increase reliability, safety, throughput including efficiency, and also lower the cost of the infrastructure needed to conduct the process.

### SUMMARY OF THE INVENTION

In a general aspect the present invention provides a method of digitising a reaction method, and more preferably a method of digitising a reaction sequence, optionally also including the preparation of a target product. The method preferably includes the use of digital and physical reaction modules for inter-engagement in a multistep synthesis. These reaction modules may be generated from a library of standardised digital models for the reaction modules, which may be used and adapted as required.

In a first aspect there is provided a method for digitising a method of synthesis, such as chemical synthesis, the method comprising the steps of:
(i) identifying a method of synthesis, such as chemical synthesis, for a target product, which may be a multistep method of synthesis;
(ii) establishing a process sequence for the method of synthesis, which process sequence is a collection of chemical and/or physical steps within the method of synthesis;
(iii) translating the process sequence to a digital model of the method of synthesis, which digital model comprises a digital description of the chemical and/or physical steps within the method of synthesis.

The method of synthesis is preferably a multistep method of synthesis, such as having three or more reaction steps in a sequence. Here, the chemical steps may be interspersed with one or more purification steps.

In step (ii), the process sequence may be the collection of chemical and or physical steps within the method of synthesis.

In step (ii), the physical steps may include one or more steps selected from the group consisting of admixture of materials for use in the reaction, heating, cooling, degassing, irradiation and saturation.

The method of the invention preferably comprises the additional steps set out below:
(iv) designing a digital reactionware for the method of synthesis, where the digital reactionware provides a digital reaction module for each step in the process sequence, and the digital modules are digitally interconnected for the digital production of the target product;
and (v) optionally generating a physical reactionware from the digital reactionware, where the physical reactionware has a module for each step in the process sequence;
and further optionally (vi) performing a method of synthesis in the physical reactionware for the production of the target product.

In step (iv), the digital reactionware may be designed using common digital components from a digital reactionware library.

The digital reactionware may include a digital security device that codes for a physical feature in the physical reactionware, which feature is important or essential for obtaining the target product, or for obtaining the target product in a form, such as yield or purity, that is suitable for use.

The security device may relate to a feature of a reactionware, such as a feature of a reactionware, that is prepared (introduced) by 3D printing or injection moulding. Additionally or alternatively, the security device may relate to a part that is to be inserted into a reactionware, for example during a pause in the printing or moulding processes.

This digital encryption of the physical features of the device form a publicly verifiable blockchain, or other form of public-private key for allowing only authorised manufacture of the chemical, and to ensure the correct target product is made.

In step (v), the physical reactionware may be prepared, at least in part, by 3D printing according to the design in the digital reactionware. Alternatively, the physical reactionware may be prepared, at least in part, by injection moulding according to the design in the digital reactionware.

In one embodiment, the physical reactionware is a unified (monolithic) piece of reactionware comprising a plurality of modules. The modules are connected, to allow material, such as fluid, transfer between modules.

Alternatively, the reactionware may comprise a plurality of interconnectable pieces, where each piece comprises one or more, such as one, module. The modules may be directly connectable, or indirectly connectable, to allow material, such as fluid, transfer between modules.

In a further aspect of the invention there is provided a method of preparing a digital reactionware, the method comprising steps (i) to (iv) as described above.

In yet a further aspect of the invention there is provided a digital reactionware, which reactionware may be provided on a computer or a memory device. The digital reactionware may be obtained or obtainable from the method of preparing a digital reactionware according to the present invention.

In yet a further aspect of the invention there is provided a method of preparing a physical reactionware suitable for use in the production of a target product, the method comprising steps (iv) and (v), such as (iii) to (v), such as (ii) to (v), such as (i) to (v), as described above.

In a further aspect there is provided a reactionware that is obtained or obtainable from the method of preparing a physical reactionware according to the present invention.

A method of the invention may comprise the step of chemically implementing a digital reaction process for the production of a target product.

In a further aspect of there is provided a product that is obtained or obtainable from the method of chemically implementing a digital reaction process for the production of the product. This aspect of the invention may comprise steps (v) and (vi), such as (iv) to (vi), such as (ii) to (vi), such as (i) to (vi) as described above.

In yet a further aspect of the invention there is provided a kit for preparing a target product, wherein the kit comprises a reactionware obtained or obtainable by the methods of the present invention, together with one or more reagents, catalysts and/or solvents, and optionally together with a set of instructions for preparing the target product using the reactionware together with the reagents, catalysts and/or solvents.

These and other aspects and embodiment of the invention are described in further detail below.

### SUMMARY OF THE FIGURES

FIG. 1 is a schematic representation of the translation of a multistep synthesis of a target product D from conception through to implementation as a reaction cartridge, according to an embodiment of the invention. Reactions necessary for the synthesis are identified (A→B→C→D, top left panel) and the specific chemical and physical processes and reaction parameters necessary for each reaction are laid out (conditions i.—iii., left panel). These processes are then translated into bespoke reaction modules designed to accomplish one or more of the chemical processes identified in the previous step (top right panel). The modules are then designed as 3D CAD models (lower centre panel), using libraries of module components to accommodate the required reaction parameters. These digital models can then be fabricated to produce either a modular or monolithic implementation (lower right panel) of the process.

FIG. 2 shows the reaction schemes for the production of (±)-baclofen (top), lamotrigine (middle) and zolimidine (bottom), in a comparison of glass reactors with polypropylene (PP) reactionware with reaction yields for each step given for the optimized synthetic route (reaction yields for the PP vessels are given in parentheses). Single (top right) or double (bottom right) chambered polypropylene reaction test cartridges were used. TBAF, tetrabutylammonium fluoride. THF, tetrahydrofuran.

FIG. 3 is schematic showing a parameterized approach to the design of individual process modules according to an embodiment of the invention where digital libraries of module components (top) can be easily assembled to produce a wide range of module geometries dictated by the specific process and reaction parameters (e.g. solvent volumes, number of inputs/outputs, etc.) (bottom). Hydrophobic filters for phase separation are shown in red, whilst fritted glass filters are shown in blue. DCM, Dichloromethane.

FIG. 4 shows (top) a schematic for the synthesis of (±)-baclofen in a series reaction cartridges, with the conceptual synthetic procedure for the synthesis of (±)-baclofen under the conditions described in FIG. 2., showing the necessary processing sequence to effect this synthetic pathway; and these processes may be then split into modules a-e (indicated by grey boxes in the process sequences of the (top)) which were translated into a digital design (middle left, schematic) and finally fabricated as either a modular (middle right, photographic image) or monolithic (bottom left, photographic image) implementation. A partially fabricated monolithic cartridge is also shown indicating the placement of non-3D printed components and internal fluidic pathways (bottom center and right, photographic images). Both modular and monolithic cartridges are shown with Luer taper-compatible valves for interfacing with external fluidic inputs and pressure/vacuum lines. The scale bar in each photograph is 3 cm.

FIG. 5 shows a schematic showing the general premise of the invention, whereby digital reaction modules are generated as coding for a reactionware that may be obtained by, for example, 3D printing the reactionware to the digital design.

FIG. 6 shows the OpenSCAD renderings of (a) a reactor module defined by the following variables: reactor(ed=40, id=28, h=50, flat_bot=true, open=true, adaptor_top=true, port_bot=true, adaptor_bot=true); creating an open reactor of height 50 mm, external diameter 40 mm, internal diameter 28 mm, with a flat bottom and a siphon with space for an adaptor as an output, and (b) a transfer tube module defined by the following variables: cannula(vio=40, len=10, d=3, t=3); creating a tube with 10 mm length, with an internal pipe diameter of 3 mm, and 40 mm vertical spacing between the input and the output.

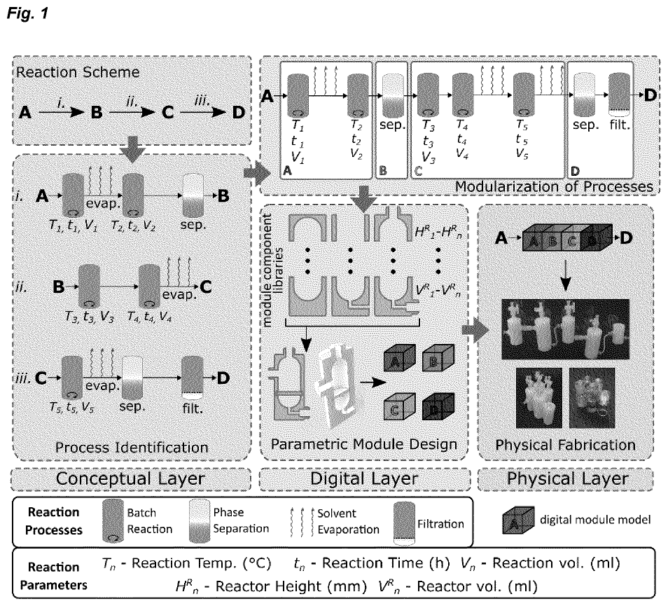

### DETAILED DESCRIPTION

The present invention provides a process for digitising a reaction process, and also a process for preparing a target product comprising the step of physically and chemically implementing a digital reaction process.

The present inventor has developed a methodology for the translation of bench-scale synthesis procedures into a step-by-step workflow which may be used to create digital designs for custom reactionware, and these may be fabricated on demand, for example using 3D printing technologies.

The large-scale manufacturing process of complex fine chemicals, such as active agents, is augmented by the present invention, which provides distributed, point-of-use manufacturing in self-contained modules (or cartridges), requiring limited user interaction to produce the target products on demand.

Here, the methods of the invention move beyond the preserve of industrial manufacturing and prototyping applications (see, for example, 5), to develop a new relationship between the design, manufacture and operation of functional devices (see, for example, 6-11).

In the preferred embodiments the method of the invention also advantageously exploit the use of 3D printing in the automation of the chemical sciences (see, for example 12-15).

The methods of the invention, in contrast to both large and medium scale traditional chemical manufacture, and also to the use of continuous-flow and microreactor approaches (see, for example, 1, 16, 17), allows for the distribution of chemical intermediates and solvents rather than the complex products themselves. These intermediates can then continue to benefit from the economies of scale brought by traditional manufacturing processes whilst complex products with short shelf lives, or lower and more distributed demand can be produced locally. This has added benefits in terms of manufacture of the final products as the synthesis of smaller quantities is inherently safer than large scale processes and poses less risk to both operators and infrastructure.

Further, the translation of these synthetic approaches into a digitally defined format, where the reactor design and, eventually, an automated synthesis procedure are encoded, could allow the digitization of all chemical products into a very low cost manufacturing format.

The invention may therefore allow large numbers of active agents to be made available as they can be brought back into production on a small scale by the fabrication and use of the appropriate cartridges.

The present inventor has previously described in WO 2013/121230 methods for the preparation of reactionware for use in performing methods of synthesis. Here, a 3D printer was used to construct bespoke reactionware for a particular reaction. The reactionware could be provided with a reaction chamber, which is a space for performing the target reaction, together with supply channels in fluid communication with the reaction chamber, for supplying material to and removing material from the reaction chamber.

The 3D printer could be suitably programmed to provide a reactionware of desired scale and structure, and suitable programming of the print sequence, with strategic pauses in the print procedure, allowed the introduction of elements into the reactionware, for example structural features for integration with an analytical device or control elements for controlling the reaction within the reaction chamber. Thus, the printing methods permitted the introduction of an optically transparent analytical window for viewing of the reaction chamber contents, as well as electrodes for contacting the reaction mixture within the reaction chamber.

However, WO 2013/121230 does not describe the digitization of a multistage synthetic procedure from a traditional synthetic route into an on-demand printed architecture. In particular, WO 2013/121230 does not suggest the development of fluidically-linked modules incorporating both reaction modules and purification modules for the preparation of a target product. Thus, WO 2013/121230 does not describe the use of architectural features to direct the synthesis, for example there is no mention of unit operations for reagent processing, mixing, and extraction.

US 8,934,994 describes methods and apparatus for automated fabrication, including the automated preparation of chemical products. The methods involve the use of a computer system to automatically predict the types of materials that could and should be used to prepare a desired product.

The method may require the computer system to request additional information from a user, such as commands, to allow for the completion of a particular synthesis. Thus, the computer system may not be sufficiently programmed with a complete description of a synthesis. Therefore, all the process steps for a synthesis are not recorded digitally, and the system requires user intervention to supply missing process information.

Although, US 8,934,994 discusses the preparation of chemical products, many other products are also mentioned, and the patent is not particularly focussed at chemical synthesis. Thus, many of the comments in U.S. Pat. No. 8,934,994 are general in nature and there is not detailed description of a process that would allow a chemical reaction to be translated into a digital model, particularly within a multistep process.

US 2016/147202 describes for processes for running a simulator of chemical reactions, and specifically a simulator for naphtha generation in an oil processing procedure. The focus is entirely on performing reactions in silica with the aim of identifying improved reactions, which reactions can then be performed in vitro.

Although US 2016/147202 appears to describe a digital model that includes reactor configuration and processing information, it does not seem as if the digital model is subsequently used to directly generate a physical embodiment of that model. In the present case a model is used to generate a reactionware, for example by operation of a 3D printer. US 2016/147202 uses a digital model to provide information about an optimised reaction scheme, and this information may be used to adapt reaction systems that are already in place.

Kitson et al. (Beilstein), which is an earlier publication from the present inventor and others, describes the autonomous synthesis of ibuprofen using a 3D printer-based robot. There is a description by the authors of a method for preparing a reaction vessel by 3D printing from a digital code, and the subsequent performance of a reaction in that reaction vessel using reagents which are delivered according to a protocol set out in a source code. The journal article does not describe in detail how the digital code is generated, nor how it might be adapted to bring about real world changes.

Kitson et al. (Chemical Science) and Dragone et al. are other publications from the present inventor and others, and they are related to the work described in WO 2013/121230. In common with WO 2013/121230, these journal articles do not describe the digitation of an entire reaction process, including the chemical and physical steps necessary for the preparation of the reaction product. Although the articles describe the use of the 3D printer to deliver a regent or catalyst, there is no complete description of the reaction process in a digital model.

In the present case the invention provides a method of taking a known reaction sequence that has been performed in the traditional manner, in glassware in a laboratory fume hood, and develops a digital coding for the reaction sequence, which encodes the physical parameters of the reactionware in a modularized form, which encompasses both the reaction steps in the reaction sequence as well as the processing steps in the reaction sequence, including most preferably the purification steps, and optionally also preparative steps for the assembly of reagents optionally together with catalysts and solvents.

In this way, a reaction sequence generated by a skilled chemist for the synthesis of a target product can be reduced to a digital coding. This coding can be used to spatially and temporarily reproduce the synthesis in a modular format allowing a non-skilled chemist to access the target product as required, and without significant burden.

The method of the invention comprises the step of translating a reaction method, which may be a known reaction method, into a digital sequence of chemical and/or physical steps. This sequence is referred to as the process sequence as it describes each of the chemical and physical steps that is required to take a starting material to the target product.

The method of the invention involves establishing a process sequence for the method of synthesis. This may be the simple identification of a process from a literature description of a known reaction method, such as to be found in the experimental section of an established journal article, or patent application, or the supplementary information supporting the former.

In an alternative embodiment, the process sequence may be established by performing the method of synthesis and noting the chemical and physical steps that are needed to yield a target product.

A description of the process steps is thereby established, and this may be subsequently translated into a digital description of the process steps.

A chemical step generally refers to a chemical reaction which yields an intermediate or final product from the reaction of one or more reagents.

A physical step generally refers to a physical step which does not encompass a chemical reaction. Within the present case, the physical step typically refers to a purification step which requires physical manipulation of a reaction mixture to at least partially purify a target intermediate or final product from other component in the reaction mixture. For example, a physical step may be the removal of a reaction solvent, for example by evaporation.

Here, the physical step may include heating or the application of a reduced pressure environment to the reaction mixture, optionally together with heating or cooling. The physical step may be a filtration step to separate insoluble components from soluble components. In a further example, the physical step may be a phase separation step, such as to separate immiscible fluids, most usually organic and aqueous phases, where one of the phases holds a target intermediate or final product, and that phase is collected.

The method of the invention includes the step of preparing a physical reactionware according to the design of a digital reactionware. The physical reactionware is the implementation of the digital design that will allow for the production of a target product with appropriate supply of reagents, solvent and catalyst, where needed, together with applied physical inputs, such as heat, light and pressure inputs into the reactionware.

The methods of preparing a reactionware described in WO 2013/121230 may be usefully used and adapted in the methods of the present case where the preparation of a reactionware is required.

Advantageously, WO 2013/121230 also describes the use of the 3D printer to deliver at least one of a reagent, solvent or catalyst to the reactionware.

The methods of preparing a reactionware in the present case are not limited to the methods described in WO 2013/121230. Whilst WO 2013/121230 focussed on the use of 3D printer technology to prepare a reactionware, the present case may also make use of other techniques for preparing a reactionware that offer the use a large degree of design freedom. For example, injection moulding techniques may be used to prepare reactionware in the present case.

WO 2013/121230 describes the manufacture of a reactionware suitable for performing a two-step and a three-step reaction sequence. There, the reactionware was provided with two and three reaction chambers respectively.

Method of Synthesis

The present invention provides for the digitisation of a method of synthesis for a target product, such as a multistep synthesis.

The method of synthesis is not particularly limited. However, the methods of the invention are particularly well suited to allowing the preparation of products that are difficult for unskilled users to prepare using standard and complex chemical techniques. The methods of the invention are also well suited to preparing target products that have a short life, for example owing to instability, such as present in radio-labelled or light-sensitive products. The methods of the invention allow for the production of target products on demand, as and when the particular product is required, and without needing the intervention of a skilled chemist.

The method of synthesis may be an organic synthesis, but the method of the invention are not limited to these, and methods of inorganic synthesis and biological synthesis are also contemplated by the invention.

The target product may be an active agent, such as a pharmaceutically active agent. In other embodiments, the target product may be a label, such as a label for a biological compound.

Establishing Process Sequence

The methods of the invention include the step of establishing a process sequence for the method of synthesis. Here, the process sequence is the collection of chemical and/or physical steps within the method of synthesis that leads to the target product from the starting materials. Thus, the process sequence is series of practical steps that need to be undertaken to allow for the production of the required intermediates, and optionally to allow for the at least partial purification of those intermediates for subsequent reaction in the synthesis. The process sequence also encompasses the isolation of the target product, such as substantially free from impurities, and the provision of the target product in a form that is suitable for use, such as in a formulation, for example a pharmaceutical formulation.

The process here may be a conceptual exercise where a skilled chemist, or a biologist, where appropriate, analyses the method of synthesis and identifies the individual steps in the method of synthesis.

The identification of steps within the method of synthesis may be made from an undertaking of the synthesis by a skilled person, for example using traditional glassware in a fumehood. The skilled person records the practical steps undertaken for use in the subsequent translation step into digital code.

Alternatively, the steps in the synthesis may be identified from a detailed description of the method of synthesis in a scientific report. Here, a skilled person may look to the detailed descriptions provided in journal articles, or their associated supplementary information, patent publications, such as applications, or methods of synthesis reported as standards in industry.

In establishing the process sequence, the skilled person has in mind the chemical reaction steps in the synthesis, whereby a compound is prepared from one or more precursor reagents. Such transformations form the basic framework of the process sequence, and the nature and order of those steps determines the product that is produced in the synthesis. For each chemical reaction the skilled person will identify or determine what chemical and physical inputs are required to allow for a particular compound to be formed.

The skilled person will also identify or determine the physical steps within the method of synthesis that are necessary, for example, for the at least partial purification of compounds, such as intermediates and the target product. Such steps are typically necessary in all syntheses to ensure the conversion of intermediate compounds to the final product, and to ensure that the target product is provided in a form that is suitable for use. The purification steps may be performed to at least partially separate a target compound, such as an intermediate or the target product, from one or more of reagents, catalyst, solvent, side products, which may include undesired isomers. Typical purification steps include solvent phase extraction, precipitation, filtration and solvent removal, amongst others.

The physical steps also encompass those practical steps that are necessary for the preparation of reagents, including intermediate compounds, catalysts and/or solvents for use in a chemical reaction. Thus, the physical steps may encompass admixture steps, where material for use in the reaction are brought together. The physical step may include practical steps such as heating, cooling, degassing, irradiation or saturation steps that prepare material for use in the reaction.

The process sequence will be established and typically comprises a series of chemical reaction steps, which may be two or more, such as three or more, such as four or more, chemical reaction steps in a sequence. The chemical reaction steps may be, and typically are, interspersed with one or more purification steps.

Translation into Digital Model

The methods of the invention may include the step of translating the established process sequence into a digital model of the method of synthesis. The digital model is a digital description of the chemical and physical

The process sequence is recorded in digital form as a coding of the individual steps and their interrelationship in the process sequence. Thus, recorded in digital form, are the chemical and practical steps that are essential to obtaining the target product.

The step of translating the process sequence may involve data entry into a computer to record the processes of the method of synthesis. Such data entry may be manual data entry, for example by a skilled chemist, entering pertinent process information into a computer. Alternatively a computer may be used to analyse reported methods of syntheses and may provide these in a suitable digital format for later use.

The digital model of the process sequence records individual steps within the process as digital modules. The digital modules are individual digital elements

The process sequence may be stored in a computer or an accessible memory device for recall by a user.

Development of Digital Reaction ware

The method of the invention provides for the development of a digital reactionware from a digital model, and a digital reactionware that is obtained and obtainable from the development of that digital model.

The digital reactionware provides a series of process modules that are developed from the individual modules established in the digital model of the method of synthesis.

A process module is a digital component forming a part of a digital reactionware, where an assembly of process modules for each of the process steps provides a digital system for the preparation of the target product.

The process module is a digitally generated component that is deemed appropriate for the performance of the process step. The provision of a process module may be based on the known suitability of a standardised process module for performing a particular task, such as a particular reaction step, or a particular physical step, such as a heating or purification step.

The modules in the digital reactionware may be individually obtained from a library of digital modules that are provided for the performance of standard tasks in a multistage synthesis. The library may be populated with modules having reaction chambers, for example, suitable for a range of applications.

The library may be populated with modules having purification components, suitable for the at least partial purification of a compound, such as an intermediate or the target compound. The module may incorporate liquid-liquid, solid-liquid, gas-liquid, gas-solid separation components. Typically, such modules are provided with filters or frits to allow separations involving insoluble material. The modules may be provided with appropriately placed outlets for the removal of solvent at a particular location within a chamber, for example as part of a phase separation and extraction procedure.

The digital modules obtained from the library may be adapted as necessary for the reaction in question, for example taking into account the scale of the reaction, which requires an appropriate sizing of the reactionware, and other factors such as the number and order of reagents and solvents to be added, which may require the provision of an appropriate number of supply channels.

The encoded digital reactionware may include security features designed to ensure the production of the target product at a desirable level and purity. The security features may also be designed to leave a mark in the reactionware or the product that provides a physical signature and guarantee of origin in the digital coding and the resulting reactionware and product.

The digital reactionware may be designed such that a particular physical feature within the reactionware is provided as a keystone to the process. The absence of this physical feature may negatively influence the production of the target product, for example to lower the yield or purity of the target product. A product that is provided in a lower than expected yield or purity may therefore be indicative of a process that has not used an authorised reactionware prepared according to an authorised digital reactionware.

The digital reactionware may be designed such a particular physical feature is provided as a marking on the surface or within the reactionware when the reactionware is prepared, such as according to the present invention.

Physical Reaction Ware

According to a further aspect of the invention there is provided a physical reactionware obtained or obtainable by the methods of the invention.

The reactionware may be obtained by 3D printing methods, such as are known to those of skill in the art. Alternatively, the reactionware may be prepared by injection moulding.

The physical reactionware used or prepared in the methods of the present case are intended for supply to an unskilled user, that is a user having little chemical knowledge and skill. The reactionware therefore provides a complete machine for use in the preparation of a target product, and the user need simply supply the relevant reagents, solvents and catalysts. Here, the reagents, solvents and catalysts may be provided in pre-packaged forms for connection into the reactionware, in a manner similar to the way in which coffee capsules are provided for connection into a coffee machine.

There is no particular limitation as to the composition of the reactionware structure: this will be dictated in part by the materials that may be used in the on-demand fabrication of reactionware, such as might be used within a 3D printer. Also, the material of the reaction will be dictated by the reaction chemistries that are to be performed in the reactionware to achieve the target product. Accordingly, the materials for the reactionware are substantially unreactive when exposed to the reagents, solvents and catalysts for use in the methods of synthesis.

As described herein, the reactionware is designed digitally to allow for the performance of the desired reaction methods. The reactionware typically consists of a plurality of modules that are connectable, either directly, such as in a monolithic unit, or indirectly as separate units that are joined by feed lines for the transfer of material, such as fluids.

A module is a part of a reactionware for conducting a step in the reaction process, such as a chemical or physical process.

A module may be provided with a reaction space for the performance of a chemical reaction. The reaction space may be provided with one or more feed channels for supply of material to the chamber. Typically these are provided above the reaction space for gravity feed into the chamber. The reaction space may be provided with one or more transfer channels for supply of material from the reaction chamber and for transfer to another module.

The digital design of the reactionware allows for design freedom in the size and the architecture of a module, and this extends to the components of the module, such as the reaction space and the supply and transfer channels. Their dimensions and structure are as required for the preparation of intermediate compounds and the product compound in the method of synthesis.

A reactionware comprises a plurality of modules, which are interconnectable and preferably interconnected, for example with fluid channels. Modules within the reactionware may comprise reaction spaces for performing chemical reactions. The reactionware of the invention preferable comprises a plurality of such reaction modules, with each reaction space provided for a separate reaction in a multistage synthesis.

Modules in the reactionware may be provided for purification of reaction mixtures, and such purification modules are typically provided in fluid communication with and downstream of a reaction module. A purification module may provide at least partially purified material to a downstream reaction module, with which it is in fluid communication.

Not every reaction module need be provided with a downstream separation module. Thus, it is not necessary to perform a purification step after each reaction step. However, whether or not a purification module is needed will depend upon the reaction sequence.

The reactionware is adapted for communication, such as connection, with vessels, such as cartridges for the supply of one or more reagents, solvents and catalysts.

Each module may be provided with suitable connectors for connection to a vessel. It may be the case that only certain modules within the reactionware have such connectors, as is the case, where only those modules require the supply of one or more reagents, solvents and catalysts.

The reactionware may also be provided with channels for the supply of gas, for example inert gases for providing an inert atmosphere in a reaction chamber. The supply of gas may also be used to apply positive pressure with modules for the movement of fluids in a module or between modules. Similarly, the channels may also be provided for reducing the pressure within a module, for example to permit movement of fluids in a module or between modules, or to volatise components in a module for its removal from the modules.

A connector for the reactionware in the present case may be Luer-style taper, such as a Luer-slip.

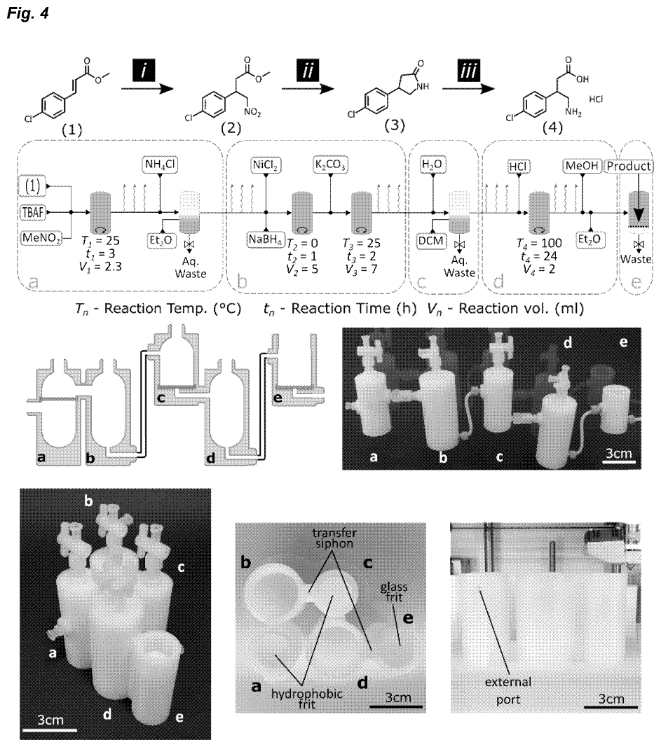

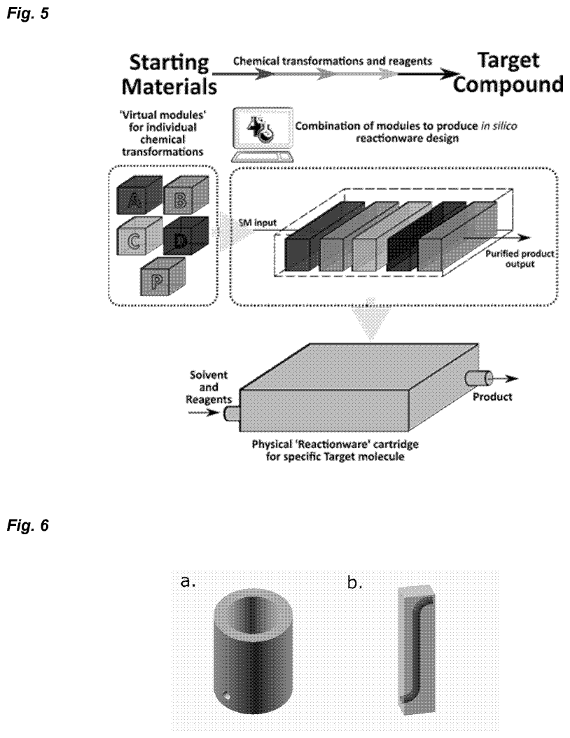

Such connectors may also be used for the connection of modules in a reactionware. Suitable fluid supply lines may be provided to interconnect modules. In other embodiments, such as where a plurality of modules are provided in a unitary piece, suitable channels may be provided within the unitary piece to interconnect the modules. A monolithic reactionware with embedded flow channels is exemplified in the worked examples of the present case (see FIG. 4). A reactionware comprising a plurality of separate modules is also exemplified, with the separate modules linked by supply lines connected to outlets and inlets that Luer tapers provided on each module (see also FIG. 4).

Uses

The present invention also provides for the exploitation of a digital reactionware.

A digital reactionware may be provided on a computer, or on a memory device such as a disk or memory card. The digital reactionware may be provided remotely, for example in the cloud, and may be readily accessible via the internet or a portal, such as a secure portal.

The digital reactionware may be obtainable from a remote source and may be saved locally, for example to a computer, such as one that is linked to a 3D printer and optionally also to reagent dispensers and analytical devices for reaction monitoring.

The digital reactionware may be converted into a physical form by preparing a reactionware according to the digital model for the reactionware. As described herein, a reactionware may be prepared, at least in part, by 3D printing or by injection moulding.

The present invention also provides a method for modifying a digital reactionware for optimisation of a method of synthesis, the method comprising the steps of preparing a reactionware according to the invention, and subsequently performing a method of synthesis according to a method of the invention. The product of the method of synthesis may be analysed, and the digital model of the method of synthesis and the digital reactionware for the method of synthesis may be revised based on the analytical results. A physical reactionware may then be prepare according to the revisions to the digital reactionware, and a method of synthesis may be performed in the revised physical reactionware, and the product of the revised synthesis may be analysed as before, for the purposes of identifying features, either chemical or physical encoded features, in the digital model that provide both positive and negative influences on the outcome of the reaction. The digital model and the digital reactionware may be optimised in silico to positively influence the outcome of the reaction.

The optimisation methods may also reveal features of the reactionware that are important in determining the reaction outcome, such as the amount and purity of the target product. These features may be identified as security features of the reactionware, that are encoded within the digital model and the digital reactionware. In the absence of these security features the reactionware cannot function to provide the target product in the desired amount and desired purity.

Kits

The present invention also provides a kit for use in the preparation of a target product.

The kit comprises a reactionware obtained or obtainable by the methods described herein, together with, and optionally together with one or more reagents, catalysts and/or solvents, and optionally together with a set of instructions for preparing the target product using the reactionware together with the reagents, catalysts and/or solvents.

The kit is supplied to a user, who may be unskilled as a chemist, and following a set of instructions, the user may add reagents, catalysts and/or solvents according to a set procedure, optionally together with the application of heating, cooling or light, for example, to give the target product.

Thus, the kit may be provided with many, such as all, of the materials necessary to form the target product. It may not be necessary to provide all the materials, as some reagents, such as water, may be readily available to the user. However, in some embodiments, all the necessary materials are provided with the kit in order to ensure an appropriate quality of reagents, catalysts and/or solvents.

The instructions to the use may be provided as a series of instructions in printed forms, such as paper. The instructions may also be accessible from the internet, and may be displayed on a computer or a personal electronic device. The instructions have a series of instructions for using the reactionware together with the relevant materials, such as reagents, catalysts and/or solvents.

In another aspect of the invention there is provided a kit comprising one or more reagents, catalysts and/or solvents, and optionally together with a set of instructions for preparing a reactionware for use in the preparation of a target product, and a set of instructions for preparing the target product using the reactionware together with the reagents, catalysts and/or solvents.

This kit may also contain materials for the preparation of the reactionware, for example materials for use in a 3D printer or materials for use injection moulding.

The kit may be used to prepare a target product, first by preparing the reactionware according to a method of the present invention, and the prepared reactionware may then be used together with the reagents, catalysts and/or solvents to prepare the target product.

The kit may further comprise parts for addition into the reactionware. As described herein, a reactionware may be provided with parts for insertion into the reactionware to assist reaction performance, to assist in reaction analysis or to assist in purification, amongst others.

Other Preferences

Each and every compatible combination of the embodiments described above is explicitly disclosed herein, as if each and every combination was individually and explicitly recited.

Various further aspects and embodiments of the present invention will be apparent to those skilled in the art in view of the present disclosure.

“and/or” where used herein is to be taken as specific disclosure of each of the two specified features or components with or without the other. For example “A and/or B” is to be taken as specific disclosure of each of (i) A, (ii) B and (iii) A and B, just as if each is set out individually herein.

Unless context dictates otherwise, the descriptions and definitions of the features set out above are not limited to any particular aspect or embodiment of the invention and apply equally to all aspects and embodiments which are described.

Certain aspects and embodiments of the invention will now be illustrated by way of example and with reference to the figures described above.

Experimental and Results

The following examples are provided solely to illustrate the present invention and are not intended to limit the scope of the invention, as described herein.

Discussion of Exemplified Syntheses

As a proof of principle, present herein is a process by which the traditional laboratory scale synthesis of a commercially available active agent can be translated into the design of an integrated cartridge. Here, all the reaction steps and intra-synthesis purification processes are encoded into the 3D architecture of a reaction cartridge so that the chemical reactions, work-ups and purification are done with minimal user intervention and exposure automatically. The process is demonstrated in the full synthesis of the anti-convulsant (±)-baclofen.

Whereas traditional laboratory syntheses will take place mostly in glassware, the present work uses polypropylene (PP) as a basic structural material for the fabrication of the cartridges. It has been found that this polyolefinic material, whilst demonstrating a robust range of chemical compatibility for traditional synthetic organic reactions, is also a suitable substrate for 3D printing applications (see Kitson et al. 2013, 2014 and 2016 (J. Org. Chem.)). This gives the best balance of chemical resistance and material properties for 3D printing.

Generally the first step in the design process is testing the reactions necessary for compatibility in the reactor materials. Future iterations of the concept can expand on the materials and fabrication processes available for the reaction modules to further develop the range of chemistries feasible in this system, using, for example, perfluorinated polymers to increase the chemical resistance of the module.

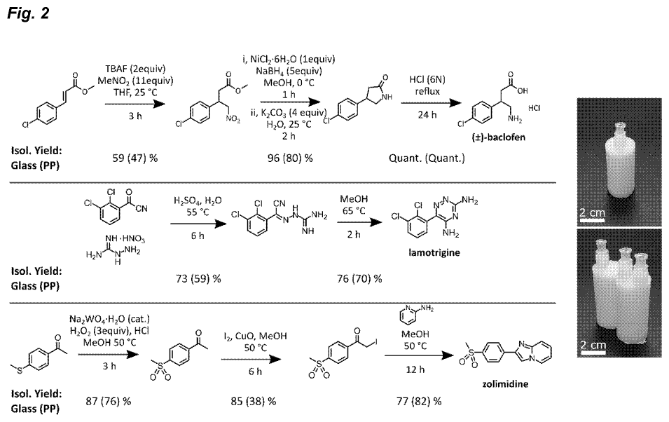

In order to demonstrate the feasibility of incorporating a basic structural material, such as PP, into reactors for the production of active agents, a number of reactions were performed which lead to such targets in test modules fabricated from PP (see FIG. 2). Reactions for the synthesis of three different APIs were performed: the central nervous system inhibitor (±)-baclofen (see Da Prada et al.), the anti-convulsant lamotrigine (see Fitton et al.), and the gastroprotective agent zolimidine (see Almirante et al.).

As can be seen from the experimental methods described below, all of the reactions tested were observed to work, but with slightly lower efficiency in PP reactors than in traditional glass reactors, due to physical loss of material on the relatively rough PP surface hampering product recovery. Surface roughness is inherent in the 3D printed process; however, use of other, as yet undeveloped, materials or different manufacturing techniques could reduce this issue. The zolimidine reactions, particularly the copper catalyzed iodination reaction, experienced a pronounced reduction in efficiency, compared to (±)-baclofen or lamotrigine. It was thought that this was due to side reactions of the iodine with the polypropylene. These issues highlight that the process of translation from glassware must take into account both the physical and chemical properties and limitations of the reactor substrate (see Kitson et al. 2013). For this reason, the first two syntheses were selected for further development into reaction cartridges to explore the concept.

Once the processes needed for the reaction sequence are identified, the combined continuous protocol is mapped onto the 3D digital designs for the target-specific cartridge. The sequence of processes is split into a series of modules with each representing a telescoped series of processes which can take place in a single chamber of the 3D printed system. Each process module is then created as a digital model which can be manipulated to provide the correct physical dimensions necessary for the reaction scheme. The 3D models of the cartridges used in this study were created with OpenSCAD software, an open-source framework for CSG (constructive solid geometry) modelling that allows a highly flexible and configurable approach to create versatile libraries of components as re-usable pieces of code. Once defined, these pieces of code can be manipulated by the software, allowing the generation of complex reactor geometries with minimal human input. For example, in this study we designed a module library consisting of interchangeable top and bottom components with varying features that can be easily combined to produce reaction vessels with different shapes and features.

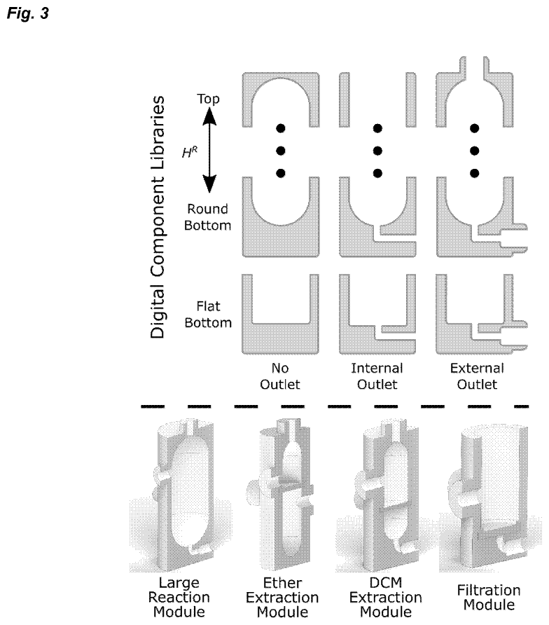

From a single line of code, an entire module can be created, with 18 different shapes available (i.e. three different tops and six different bottoms can be selected, see FIG. 3). The modules were designed around simple chambers where each reaction/process could be performed in as close a manner as possible to the way it would be carried out using traditional batch chemical techniques, easing the transition between published synthesis in glassware and ‘cartridge’ synthesis. Typically, a standard module has an opening on the top of the wall of the chamber for transfer of reaction mixtures from previous modules, and an opening at the bottom of the chamber for expelling material from the module subsequent to the completion of the desired process.

The transfer of material between modules may be facilitated by a further opening in the roof of the compartment which can be used to supply a gas flow, such as increased pressure, to force the reaction medium out of the chamber via the outlet at the bottom. The opening at the top otherwise equalizes pressure throughout the device to prevent the premature transfer of material, and also allows for application of vacuum to remove and exchange of solvents. These modules can then be combined in sequences by use of further components of our module library such as siphon tubes for the transfer of material from one reaction module to another.

Once a reaction chamber is created, new features can be added by subtracting or adding shapes to the module. For example, a filtration device can be made from a module with a top input, a round bottom with a port, and a glass filter. To achieve this feature, a cylindrical model conforming to the dimensions of the physical filter to be inserted is created and subsequently subtracted from the model of a reaction chamber producing a void space in the model into which the filter fits. Phase separation modules were achieved in a similar manner using hydrophobic frit inserts which effectively separate organic and aqueous phases for product extractions. In keeping with the desire to design synthesis cartridges which can be produced outside traditional manufacturing regimes, 3D printed reactors—reactionware—for synthetic chemical applications are used to prototype the physical reactors (see Symes et al.; Kitson et al. 2016 (Nat. Protocols)). 3D printing based fabrication approaches have the added advantage of being intimately linked to the design process.

Fabrication of a modular system was carried out on the low-cost 3D printers Ultimaker 2/2+, although many other Fused Deposition Modeling (FDM) printers would similarly be able to print the 3D modules produced through this approach.

If the incorporation of non-printed materials was necessary during 3D printing of the final module, a pre-programmed pause in the printing process is instigated at a point just above the designed void, and the component is inserted in this space prior to the resumption of printing. Upon completion of printing the inlet/outlet ports were tapped with a ¼ inch UNF thread to allow ease of integration with the external infrastructure for performing the reaction sequences. Using standard ports allowed us to attach either standard fluidic tubing connectors such as those found in traditional flow synthesis set-ups, or widely used Luer lock adaptors. These Luer lock connectors are easily reconfigurable facilitating feedback into the design process.

The active agent selected to exemplify a complete end-to-end synthesis was the central nervous system depressant and anti-spastic medication (±)-baclofen (see Camps et al.; see Mann et al.) [RS-β-(4-chlorophenyl)-γ-aminobutyric acid] (4) (see FIG. 4), a derivative of γ-aminobutyric acid (GABA) that modulates the action of this central inhibitory neurotransmitter (see Da Prada et al.). This target was chosen as an example to demonstrate the fact that even relatively short syntheses require a disproportionately larger set of chemical processing steps required to effect the full synthesis; in future it is envisioned that the synthesis of larger numbers of compounds and compound classes will greatly expand the scope of this approach.

(±)-Baclofen has found a number of uses since its first reported synthesis and is currently being investigated beyond its traditional usage for anti-alcoholism applications in high doses (see Addolorato et al.). Many syntheses of (±)-baclofen have been published since it was first reported, often proceeding though the formation, and subsequent hydrolysis of, β-(4-chlorophenyl)-γ-butyrolactam (3). The traditional synthesis of (±)-baclofen was modified, starting from the commercially available material methyl 4-chloro-cinammate (1), and proceeding via the Michael addition of nitromethane to form 4-nitro-3-(4-chlorophenyl)butanoic acid (2), followed by nickel catalyzed reductive lactamization and subsequent acid hydrolysis to produce the final product in its commercially available racemic form as a hydrochloride salt. This three-reaction-step sequence contains 12 individual processing steps which need to be incorporated into the reactionware device to complete the synthesis (see FIG. 4).

This sequence was designed to be particularly amenable to translation into the modular/monolithic system as at each stage, the reactions are either sufficiently clean, or reaction impurities which would impinge on subsequent process in the synthesis could be readily removed by phase partition. The final product is purified through a methanol/diethyl ether crystallization which yields a crystalline solid that can be retrieved directly from the cartridge device.

Each of these processes was translated into operations which could be successfully embodied in one or more reaction/purification modules. The specific reaction modules used for the synthesis of (±)-baclofen were: (a) a combined Michael addition, evaporation and ether extraction module, (b) a combined solvent exchange and reduction module, (c) a phase separation and filtration module, (d) a combined solvent exchange and hydrolysis module, and (e) a filtration module. Individual modules were fabricated for a ‘plug-and-play’ approach to the reaction process development using Luer lock fittings to connect individual modules, and Luer taper compatible valves to interface with pressure/vacuum systems.

This design allowed testing of each individual process in isolation before the modules were combined to build up the full synthesis. Finally the module designs were ‘digitally stitched together’ using the developed CAD libraries for internal fluidic pathways to create the design for a monolithic synthesis cartridge. Once fabricated the individual modules and the monolithic cartridges were evacuated and filled with a nitrogen atmosphere to ensure an inert environment for the subsequent chemistry.

The first chamber, (a), consisted of a lower volume (4.9 mL) where the initial reaction can take place which is separated from the upper outlet by a hydrophobic frit. Reactor modules (b) and (d) consisted of a single unbroken reaction chamber (31.8 mL) with sufficient volume to accommodate the reaction volumes and extraction solvents from the previous processes prior to concentration under reduced pressure. Extraction module (c) consisted of a chamber of sufficient volume (4.7 mL) to contain the aqueous phase from the previous chamber, which had a drain at the bottom covered by a hydrophobic frit that prevented both solid material and aqueous solution from passing into the next chamber/module. The final module was a filtration module for separating and retrieving the final product. This one module could be either open to the atmosphere or enclosed as required.

During the fabrication process, chambers/modules which required stirring were equipped with a PTFE coated magnetic stirring bead (length 10 mm) to enable mixing of the contents. Each module/chamber of the monolith was equipped with a ¼ inch UNF threaded port carrying a female Luer lock adaptor which was used to introduce an inert (dry, N₂) atmosphere, or reduced pressure, into the system. The modular system was designed such that there was a single fluidic path through the reactor: flow from one chamber into the next was induced either by pressure from excess solvent, in the case of the phase separation processes, or the introduction of nitrogen pressure difference between the relevant chambers to push the reaction mixture through an embedded channel running from the bottom of one chamber to the top of the next.

Starting materials were prepared as simple solutions and transferred to the cartridge via standard Luer syringes. The cooling and heating required for the reaction sequence was achieved by the immersion of the reaction cartridge/module in an ice or sand bath respectively and the temperature required for the reactions can be achieved automatically on a stirring-hotplate. The exact sequence of operations, positioning of the module in the heating/cooling bath and time intervals necessary to complete the synthesis are outlined below.

Performing the synthesis starting from 200 mg of 1 (methyl 4-chlorocinnamate) in the manner described yielded 98 mg (39% yield over three reaction steps and 12 processing steps from 1 with ≥95% purity as determined by H PLC) of (±)-baclofen hydrochloride salt, which is more than one day's maximum dosage of the drug.

Better efficiency of reaction was achieved using lower concentrations of staring materials (using a similar cartridge at half concentration, i.e. 100 mg scale, gave a 44% yield over three steps of similar purity). Increasing the volume of the reactor as well increases the quantity of (±)-baclofen obtained (a 300 mg scale synthesis yielded 133 mg (35%) (±)-baclofen).

The integration of the reaction processing steps into the design of the modules greatly simplifies the operations required to perform the reaction sequence compared to traditional bench synthesis and simultaneously reduces the level of technical skills required to perform the process down to simple operations which do not require the specific skills of a trained synthetic chemist. Whilst the total time for the reaction sequence is around 40 hours in this case, including all intermediate operations, the workflow is constrained by the geometry of the device, so all human interaction is limited to simple interventions at specific time periods, and it should be possible to shorten the interaction time further. The use of such bespoke, single use cartridges would greatly reduce the time spent on glassware preparation, liquid handling and other ancillary tasks associated with the majority of chemical syntheses at this scale. Also, by using the geometry of the reactor to constrain the operation of the synthesis we reduce the human decision making involved in the synthesis processes making the sequence more reproducible. Given sufficient facilities several instances of the synthesis cartridge could be used at once achieving scalability by numbering—up arrays of cartridges, and using these in parallel to increase the output. As a result of the ability to parameterise and encode multi-step organic synthesis reactions with workups embedded, we envisage that a digital programmable universal heater/stirrer/solvent/reagent plug-and-play device can be constructed into which only the cartridge, unique to a given synthesis, can be plugged in.

The (±)-baclofen synthesis necessitated liquid handling and separation of reaction chambers to effect the full reaction sequence. In some cases, however, syntheses can be conducted in single reaction cartridges depending on the nature and quality of the inter-step purification required.

For example, the synthesis of lamotrigine (see FIG. 2) was achieved in a single cartridge as the intermediate material is insoluble in the reaction solvent at low temperatures. In a single, closed, filtration module, the initial reaction product could be washed and processed in-situ prior to the introduction of the solvent for the subsequent cyclization step. This is in contrast to the traditional procedure, which requires the solid product of the first step to be removed from the initial reactor to be filtered, dried and then reintroduced to a reactor for the second step of the synthesis. Performing the synthesis of lamotrigine on a 250 mg scale of starting material yields 112 mg (46% over two reaction steps) of the final product, giving an off white crystalline powder.

General Comments

Solvents and reagents were used as received from commercial suppliers unless otherwise stated. Polypropylene feedstock for 3D printing was purchased from Barnes Plastic Welding Equipment Ltd., Blackburn, UK. 3D printing was achieved on an Ultimaker 2/2+ supplied by Ultimaker. ¹H NMR and ¹³C NMR spectra were recorded on a Bruker Avance 400 MHz spectrometer at 298 K, and chemical shifts are reported in ppm relative to residual solvent (multiplicities are given as s: singlet, d: doublet, t: triplet, q: quartet, m: multiplet, with coupling constants reported in Hz). Mass Spectra were recorded on a Q-trap, time-of-flight MS (MicroTOF-Q MS) instrument equipped with an electrospray (ESI) source supplied by Bruker Daltonics Ltd. All analysis was collected in positive ion mode. The spectrometer was calibrated with the standard tune-mix to give a precision of ca.1.5 ppm in the region of m/z 100-3000. Percentage purity was assessed on a Dionex 3000 Ulitmate HPLC system comprising LPG-3400SD pump, DAD-3000 detector with a 13 μL flow cell, WPS-3000TFC analytical autosampler with fraction collector, and TCC-3000SD column thermostat, running

Chromeleon 6.8. A reversed-phase C18 column (Purospher STAR RP-18 endcapped (5 μm), 100×4.6 mm) was used.

Module/Cartridge Design and Fabrication

All reactors used in this study were designed either using OpenSCAD open source script based 3D modeling software (www.openscad.org) or FreeCAD, open source GUI based 3D modeling software (www.freecadweb.org) and printed on either a Bits-from-Bytes ‘3D Touch’ 3D printer (discontinued) or Ultimaker 2 and 2+3D printers (https://ultimaker.com/), with nozzle diameter of 0.4 or 0.6 mm using polypropylene from a local supplier. The designs for the synthesis cartridges were exported as stereolithography (.stl) files and translated into 3D printer instruction files using software appropriate for the 3D printer employed (Axon 20. Software, supplied by Bits from Bytes, for the 3D touch printer, or Cura (https://ultimaker.com/en/products/cura-software), a freely available 3D printer control software package developed by Ultimaker. These instruction files are then transferred to the 3D printer for fabrication.

Devices were printed at 260° C. on polypropylene plates to avoid warping (the use of a heating bed was unnecessary). To allow the introduction of necessary reagents, starting materials, or non-printed components the printing process was modified to pause at pre-programmed intervals during the fabrication to allow their placement (see below). Once cartridge fabrication was complete the cartridges were flushed with a suitable inert gas (dry N₂ supplied by BOC) and sealed prior to use.

Parametric Module Design

OpenSCAD libraries used for the production of digital model files were provided (digital model files not provided here). The libraries' purpose is to easily create high level objects (e.g. reactors, pipes, cannulas, etc.) which can be joined together in silico to create parametrically designed reactor/process modules. The modules/cartridges designed for this work were composed of four libraries: lib_pipe (to create straight pipes), lib_bend_pipe (to create bent pipes), lib_canula (to connect two reactors), and lib_reactor (to create reactors). This allows modules to be created by specifying the required dimensions for a limited number of reaction parameters to obtain models with the specific dimensions necessary for the synthesis being developed.

“Reactor” objects can then be modified to form the basis of a range of different modules. For example, co-axially oriented cylinders can be subtracted from main reaction volume to insert filters at defined positions, whether radial-oriented cylinders (with respect to module axis) can be subtracted from the walls of the reactor to create input holes as required by the specific synthesis requirements.

The design parameters are set out below in Table 1, for a reactor defined as reactor(ed, id, h, flat_bot=false, open=false, adaptor_top=true, port_bot=true, adaptor_bot=false)

TABLE 1
Parameters required for the generation of a reactor module

| Param- | | |
|---|---|---|
| eter | Units | Definition |
| ed | mm | Outer diameter of the reactor |
| id | mm | Inner diameter of the reactor |
| h | mm | Height of the reactor |
| flat_bot | true/ | Defines the bottom profile of the internal chamber; |
| | false | if ′true′, the chamber bottom will be hemispherical |
| | | in shape; if ′false′ the chamber will have a flat |
| | | bottom. Default set to false. |
| open | true/ | Defines whether the top of the chamber is open |
| | false | or closed. If true, the top of the reactor will be |
| | | open. Default set to false. |
| adaptor_ | true/ | Defines the presence of an input/output port at the |
| top | false | top of the chamber. If true and ′open′ is false, |
| | | creates an input at the top of the reactor. |
| | | Default set to true. |
| port_bot | true/ | Defines the presence of an input/output port at the |
| | false | bottom of the chamber. If true, create a siphon |
| | | output from the bottom of the reactor. |
| | | Default set to true. |
| adaptor_ | true/ | Defines the presence of an external port at the |
| bot | false | bottom of the chamber. If true, creates space for |
| | | an adaptor at the bottom. |
| | | Default set to false. |

Reactors (or the modules built from them) can be connected to each other using the “cannula” and “pipe” libraries. For example, the bottom output of a reactor is connected to the input (the lowest entry point) of a transfer tube. The output of the transfer pipe will then lead to the inner volume of the next module.

The design parameters are set out below in Table 1, for a cannula defined as cannula(vio, len, d, t).

TABLE 2
Parameters required for the generation of an
inter chamber cannula

| Param- | | |
|---|---|---|
| eter | Units | Definition |
| vio | mm | Vertical distance between input and output. |
| len | mm | Horizontal distance between input and |
| | | output. |
| d | mm | Inner diameter of the tube. |
| t | mm | Thickness of the walls. |

These parameters can then be defined to generate specific instances of reactor or transfer tube objects which can then be manipulated to produce the finalized cartridge devices, such as shown in FIG. 6, which is illustrative of a reactor (a) and a transfer tube (b).

Modular cartridges were 3D printed with openings to screw in standard Luer-lock connectors after tapping, and Tygon tubing (⅛ or 1/16″ external diameter) were used to connect the modules. For liquid-liquid separation modules, TELOS Phase Separators (20 mm diameter) were used. For filtration modules, 20 mm glass filters (porosity 2) were used. While designing cartridges with embedded filters, a precise clearance between the filter disc and reactor walls has to be maintained to avoid leaks. It was found that a filter enclosure diameter should be equal to the diameter of the filter +0.85 mm (as set in the software), and the enclosure height should be equal to that of the filter itself. To insert filters during the 3D printing process, Cura's “Pause at Height” plugin was used, allowing the fabrication to be automatically halted at a desired point to allow the manual insertion of the non-printed component (see FIG. 4).

Baclofen Synthesis Modules

The modules designed for the translation of the end-to-end synthesis of baclofen are as set out below. The baclofen synthesis was considered and a process sequence was established, identifying the chemical and physical steps needed to prepare the target product from the starting material, methyl 4-chlorocinnamate. This reaction sequence was then translated into a digital model for the generation of a digital reactionware.

FIG. 4 shows the reduction of the baclofen synthesis (top line) to a series of modules for undertaking the chemical and physical steps, including reaction steps, heating steps, and separation steps.

A digital reactionware was generated with a module for each of the identified steps in the digital model, and a physical reactionware was generated comprising a series of connectable physical modules.

Module (a): 1st Reaction; Concentration; Aqueous—Organic separation

This module was based on a cylindrical, closed, round bottomed chamber with three inlet/outlet openings, one at the top of the module and two on either side of the vertical walls separated by a hydrophobic frit to achieve the necessary organic/aqueous separation. The volume below the frit was 4.9 mL and the volume above the frit was 3.2 mL. This module was designed for phase separations in which the organic layer is less dense than the aqueous layer.

Module (b) & (d): Solvent removal and reactions 2 & 3

This module was designed to have a large capacity to accommodate the excess solvent required for upstream extraction processes. It was based on a cylindrical, closed, round bottomed chamber with two inlet ports; one on the vertical wall of the chamber and one at the top of the module, and an outlet ‘drain’ at the bottom of the module which consisted of a bent pipe leading from the lowest point of the internal chamber to an outlet port located at the bottom of the vertical wall of the chamber on the opposing side to the inlet port. The total volume of the chamber was 31.8 mL.

Module c: Aqueous—Organic separation

This module was based on a cylindrical, closed, round bottomed chamber with two inlet openings, one at the top of the module and one at the top of the vertical wall of the module, and an outlet ‘drain’ at the bottom of the module which consists of a bent pipe leading from the lowest point of the internal chamber to an outlet port located at the bottom of the vertical wall of the chamber on the opposing side to the inlet port. The internal chamber is divided by a hydrophobic frit placed at approximately one third of the full height of the internal chamber to achieve the necessary organic/aqueous separations. The volume below the frit was 2.1 mL and the volume above the frit was 4.7 mL. This module was designed for phase separations in which the organic layer was denser than the aqueous layer.

Module e: Filtration module

This module was based on a cylindrical, open-topped, round bottomed chamber with one inlet opening, one at the top of the vertical wall of the module, and an outlet ‘drain’ at the bottom of the module which consists of a bent pipe leading from the lowest point of the internal chamber to an outlet port located at the bottom of the vertical wall of the chamber on the opposing side to the inlet port. At the bottom of the chamber a fritted glass filter is present which is inserted during the fabrication process. The total volume of the chamber was 8.0 mL.

Baclofen Monolithic Synthesis Cartridge

Once the designs for the modules necessary for the synthesis of baclofen were tested (see above), these modules were combined in OpenSCAD, with minor modifications, to produce a monolithic cartridge for the complete synthesis of baclofen. The modules were connected to each other either by directly connecting the outlet and inlet ports designed in the original modules or via siphon channels connecting drain outlets at the bottom of module to input port at the top of the subsequent module. The orientation of the inputs and outputs were rotated around the central axis of each module as required to minimise the footprint of the final cartridge. Minor optimisations were also made based on the experience of the use of the modular system, for example the space below the hydrophobic filter in module c was removed. The cartridge was designed such that all non-printed parts were at the same height from the base of the cartridge to minimise the number of pauses required during fabrication.

Module/Cartridge Inlets & Outlets

In order to interface the synthesis cartridges with the external fluid handling of the system, each design was produced with openings suitable for tapping to produce a ¼″ UNF thread which could be fitted with polypropylene adapters for Luer-lock fittings (supplied by Cole Parmer) allowing ease of connection to external fluidics. These Luer-lock interfaces proved to be solvent tight and gas tight under the conditions of the syntheses described here.

The tapped openings could be also fitted with barbed fittings for directly connecting cartridges to external tubing, or standard flangeless fittings for interfacing with external tubing. Individual modules could be connected directly by fitting a ¼″ UNF to female Luer adaptor on one module into a male Luer to ¼″ UNF adaptor on the next module, reducing the flow path for reaction media through the modular systems.nted.

Traditional/Polypropylene Synthesis/Analysis

Below are given details of the synthetic procedure for the test reactions in both polypropylene and glass reactors conducted for each step of the three syntheses considered for application in reactionware cartridges. Diagnostic ¹H and ¹³C NMR spectra shown are taken from glassware reactions but comparison of analysis for both regimes confirmed the identity of products obtained.

Synthesis of (±)—Baclofen

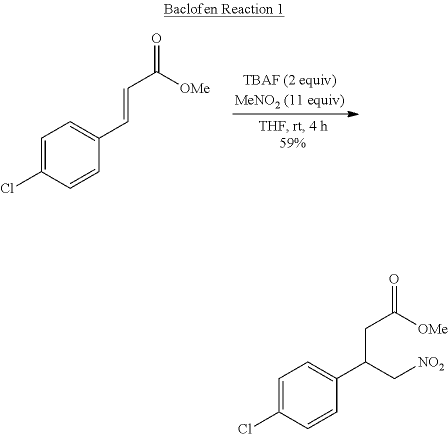 [SMILES: COC(=O)/C=C/c1ccc(Cl)cc1 ; COC(=O)CC(C[N+](=O)[O-])c1ccc(Cl)cc1]

Traditional (Glassware) Synthesis

In a 25 mL round bottomed flask, methyl 4-chlorocinnamate (1.0 g, 5.10 mmol) followed by nitromethane (3.0 mL, 56.1 mmol, 11 equiv.) were added into a 1 M solution of tetrabutylammonium fluoride in THF (10.2 mL, 10.2 mmol, 2 equiv.). The reaction was stirred over 4 hours at room temperature. Afterwards, the resulting yellow crude mixture was quenched with an aqueous solution of HCl (2 M) until a colorless solution was obtained. The solvents were removed under reduced pressure and the residual reaction mixture was purified by flash chromatography (Petrol./EtOAc 10:1 and then 4:1). The desired γ-nitro ester was obtained (771 mg, 3.0 mmol, 59%) as a colorless oil.

Polypropylene Test Reaction

In an 11 mL polypropylene reactionware vessel, methyl 4-chlorocinnamate (100 mg, 0.51 mmol) followed by nitromethane (0.3 mL, 5.61 mmol, 11 equiv) were added into a 1 M solution of tetrabutylammonium fluoride (TBAF) in THF (1.0 mL, 1.02 mmol, 2 equiv). The reaction was stirred over 4 hours at room temperature. Afterwards, the resulting yellow crude mixture was quenched with an aqueous solution of HCl (2 M) until a colorless solution was obtained. The solvents were removed under reduced pressure and the residual reaction mixture was purified by flash chromatography (Petrol./EtOAc 10:1 and then 4:1). The desired γ-nitro ester was obtained (62 mg, 0.24 mmol, 47%) as a colorless oil.

¹H NMR (400 MHz, CDCl₃): δ =7.32 (2H, d, J=8.8Hz), 7.17 (2H, d, J=8.4Hz), 4.72 (1H, m), 4.61 (1H, m), 3.97(1H, q, J=7.6Hz), 3.13(3H, s), 2.75(2H, m). ¹³C NMR (101 MHz, CDCl₃): 171.0, 136.9, 134.2, 129.5, 128.9, 79.3, 52.2, 39.8, 37.4. IR (v.): 3005, 2955, 1732, 1551, 1493, 1437, 1377, 1267, 1225, 1198, 1169, 1096, 1015, 880, 829, 733, 648 cm⁻¹. HRMS (ESI) calculated for C₁₁H₁₂NO₄N³⁵ClNa [M+Na]⁺ 280.0347, found 280.0340. Δ=2.5 ppm.

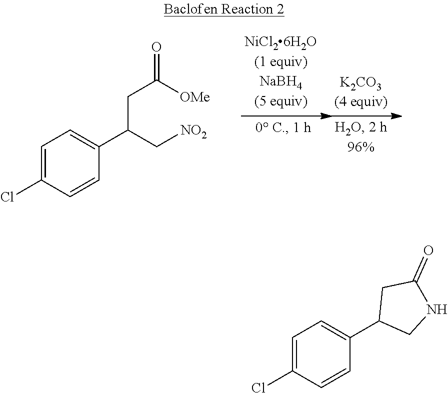 [SMILES: COC(=O)CC(C[N+](=O)[O-])c1ccc(Cl)cc1 ; O=C1CC(c2ccc(Cl)cc2)CN1]

Traditional (Glassware) Synthesis

In a 25 mL round bottomed flask NaBH4 (142 mg, 3.75 mmol, 5 equiv) was added in portions into a solution of the nitro ester (193 mg, 0.75 mmol) and NiCl₂.6H₂O (178 mg, 0.75 mmol, 1 equiv.) in MeOH (10 mL). The reaction was stirred over 30 minutes at 0° C. After quenching the crude mixture with a solution of K₂CO₃ (415 mg, 3.0 mmol, 4 equiv.) in water (5 mL) for 2 hours, the aqueous phase was extracted with EtOAc (3×40 mL). The combined organic layers were dried over Na₂SO₄ and filtered. The solvents were evaporated in vacuo to obtain the desired γ-lactam (140 mg, 0.72 mmol, 96%) as a white solid.

Polypropylene Test Reaction

In an 11 mL polypropylene reactionware vessel, NaBH₄ (59 mg, 1.6 mmol, 4 equiv.) was added in portions into a solution of the nitro ester (100 mg, 0.39 mmol) and NiCl₂.6H₂O (93.0 mg, 0.39 mmol, 1 equiv.) in MeOH (4 mL). The reaction was stirred over 30 minutes at 0° C. After quenching the crude mixture with a solution of K2CO3 (221 mg, 1.6 mmol, 4 equiv.) in water (2.0 mL) for 2 hours. The aqueous phase was extracted with CH₂Cl₂ (5 mL) and transferred into the next cartridge through the hydrophobic filter disc. The solvents were evaporated in vacuo to obtain the desired γ-lactam (60 mg, 0.31 mmol, 80%) as a white solid.

m.p. 113° C. ¹H NMR (400 MHz, CDCl₃): δ =7.32 (2H, d, J=8.0Hz), 7.19 (2H, d, J=8 Hz), 5.88 (1H, br), 3.80-3.76 (1H, m), 3.68 (1H, q, J=8Hz), 3.40-3.36 (1H, m), 2.74 (1H, dd, J=16.8, 8.8Hz), 2.45 (1H, dd, J=16.8, 8.8Hz). ¹³C NMR (101 MHz, CDCl₃): δ=177.2, 140.8, 133.2, 129.2, 128.2, 49.4, 39.9, 37.9. IR (vₘₐₓ): 3195, 2934, 2359, 2245, 2045, 2029, 1967, 1688, 1493, 1456, 1404, 1346, 1260, 1088, 1013, 887, 800 cm⁻¹.

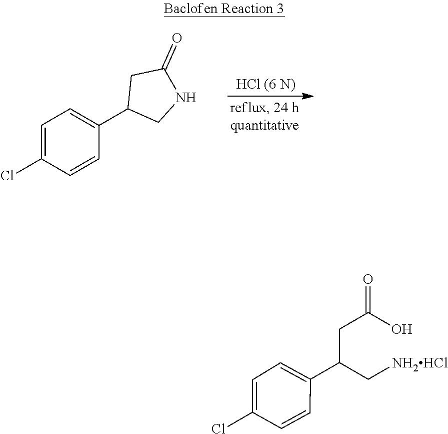

Traditional (Glassware) Synthesis

In a 10 mL round bottomed flask, the y-lactam (80 mg, 0.41 mmol) was mixed in an aqueous solution of HCI (6 m, 2.5 mL). The reaction mixture was then refluxed over 24 hours. The resulting mixture was cooled down to room temperature and carefully concentrated under vacuum to afford (±)-baclofen (101 mg, 0.40 mmol, quantitative yield).

Polypropylene Test Reaction:

In an 11 mL polypropylene reactionware vessel, the γ-lactam (32 mg, 0.16 mmol) was mixed in an aqueous solution of HCI (6 m, 1.3 mL). The reaction mixture was then refluxed over 24 hours. The resulting mixture was cooled down to room temperature and carefully concentrated under vacuum to afford baclofen (39 mg, 0.16 mmol, quantitative yield).

m.p. 192-194° C. ¹H NMR (600 MHz, D20): δ=7.46 (2H, d, J=8.4Hz), 7.36 (2H, d, J=8.4Hz), 3.47-3.38 (2H, m), 3.28-3.24 (1H, m), 2.87 (1H, dd, J=15.6, 5.4Hz), 2.77 (1H, dd, J=16.2, 9.0Hz). ¹³C NMR (150 MHz, D₂O): δ=175.4, 137.0, 133.4, 129.4, 129.2, 43.6, 39.4, 38.3. IR (vₘₐₓ): 3700, 3074, 1765, 1591, 1514, 1493, 1408, 752, 700 cm⁻¹.

Synthesis of Zolimidine

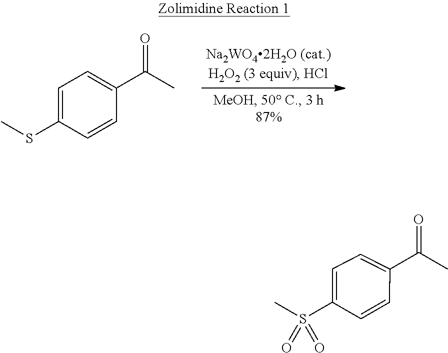

Traditional (Glassware) Synthesis

In a 25 mL round bottom flask containing 4-(methylthio)-acetophenone (250 mg, 1.50 mmol), sodium tungstate dihydrate (49.8 mg, 0.150 mmol, 10%mol), methanol (2 mL), and aqueous conc. HCl (1-2 drops) was added aqueous H₂O₂(30%, w/v, 500 pL, 4.50 mmol, 3 equiv.) and the resulting solution heated to 50° C. After 3 hours, the reaction was concentrated in vacuo and the resulting residue partitioned between an aqueous solution of NaOH (0.5 m, 20 mL) and EtOAc (20 mL). The aqueous phase was separated and extracted using EtOAc (2×10 mL). The combined organic phase was dried (MgSO₄), filtered, and concentrated in vacuo to afford 4-(methylsulfonyl)-acetophenone (258 mg, 1.30 mmol, 87%) as a white solid.

Polypropylene Test Reaction

In a polypropylene reactionware vessel containing 4-(methylthio)-acetophenone (100 mg, 0.60 mmol), sodium tungstate dihydrate (20 mg, 0.06 mmol), methanol (2 mL), and aqueous conc. HCI (1-2 drops) was added aqueous H₂O₂ (30%, w/v, 200 μL, 1.80 mmol) and the resulting solution heated to 50° C. After 3 hours, the reaction was concentrated in vacuo and the resulting residue partitioned between aqueous solution of NaOH (0.5 m, 20 mL) and EtOAc (20 mL). The aqueous phase was extracted using EtOAc (2×10 mL). The combined organic phase was dried (MgSO4), filtered, and concentrated in vacuo to afford 4-(methylsulfonyl)-acetophenone (119 mg, 0.60 mmol, 76%) as a white solid.

m.p.=132-135° C. ¹H NMR (400 MHz, CDCl₃): δ=8.17-8.10 (2H, m), 8.09-8.02 (2H, m), 3.08 (3H, s), 2.67 (3H, s). ¹³C NMR (101 MHz, CDCl₃): δ=196.8, 144.4, 141.1, 129.3, 128.0, 44.5, 27.1. IR (Uₘₐₓ): 1687, 1396, 1310, 1295, 1289, 1259, 1173, 1149, 1090, 963, 779, 748, 618 cm⁻¹. HRMS (EI) calculated for C₉H₁₀O₃S [M]⁺: 198.0351, found 198.0351. Δ=0 ppm.

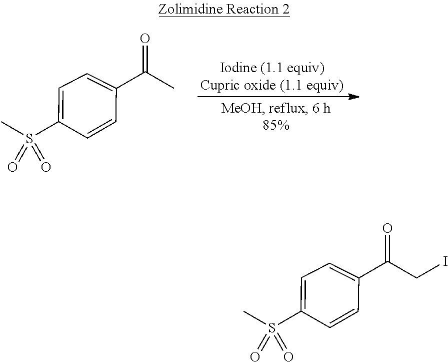

Traditional (Glassware) Synthesis

In a 25 mL round bottom flask containing acetophenone (100 mg, 0.50 mmol), iodine (141 mg, 0.55 mmol), and cupric oxide (44.1 mg, 0.55 mmol) was added methanol (2.5 mL) and the resulting suspension heated to reflux. After 6 hours, the reaction was filtered through a pad of Celite®, eluting with methanol. The filtrate was concentrated in vacuo and the residue dissolved in ethyl acetate, which was then washed with an aq. sodium thiosulfate (10%, w/v, 10 mL) solution, brine (20 mL), dried (MgSO₄), filtered, and concentrated in vacuo. Flash chromatography on silica gel (methylene chloride—ethyl acetate, 9:1) afforded α-iodoketone (122 mg, 0.38 mmol, 85%) as an off white solid.

Polypropylene test reaction

In a polypropylene reactionware vessel containing acetophenone (100 mg, 0.50 mmol), iodine (140 mg, 0.55 mmol), and cupric oxide (44.0 mg, 0.55 mmol) was added methanol (2.5 mL) and the resulting suspension heated to 70° C. After 6 hours, the reaction was filtered through a pad of Celite, eluting with methanol. The filtrate was concentrated in vacuo and the residue dissolved in EtOAc, which was then washed with an aqueous sodium thiosulfate (10%, w/v, 10 mL) solution, brine (20 mL), dried (MgSO₄), filtered, and concentrated in vacuo. Flash chromatography on silica gel (CH₂Cl₂/EtOAc: 9:1) afforded α-iodoketone (61 mg, 0.19 mmol, 38%) as an off white solid.

m.p. 134-136° C. ¹H NMR (400 MHz, CDCl₃): δ=8.17 (2H, d, J=8.4Hz), 8.08 (2H, d, J=8.3Hz), 4.39 (2H, s), 3.10 (3H, s). ¹³C NMR (101 MHz, CDCl₃): δ=191.6, 144.9, 137.6, 130.1, 128.4, 44.5, 1.0. IR (uₘₐₓ): 1674, 1319, 1298, 1267, 1151, 1009, 963, 786, 737 cm⁻¹. HRMS (ESI) calculated for C9H₁₀IO₃SNa [M+Na]⁺: 346.9215, found 346.9214. Δ=0.3 ppm.

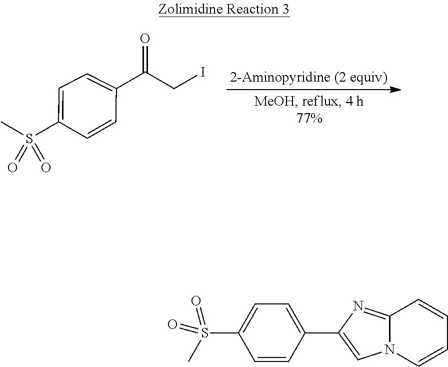 [SMILES: CS(=O)(=O)c1ccc(C(=O)CI)cc1 ; CS(=O)(=O)c1ccc(-c2cn3ccccc3n2)cc1]

Traditional (Glassware) Synthesis

In a 25 mL round bottom flask containing a solution of a-iodoketone (40.0 mg, 0.123 mmol) in methanol (5 mL) was added 2-aminopyridine (23.3 mg, 0.25 mmol, 2 equiv.) and the resulting solution heated to reflux. After 4 hours, the reaction was concentrated in vacuo and purified by flash chromatography on silica gel (CH₂Cl₂/EtOAc: 4:1 to 1:1). Zolimidine was obtained (26 mg, 0.10 mmol, 77%) as an off white solid.

Polypropylene Test Reaction

In a polypropylene reactionware vessel containing a solution of α-iodoketone (40 mg, 0.13 mmol) in methanol (3 mL) was added 2-aminopyridine (24.0 mg, 0.25 mmol, 2 equiv.) and the resulting solution heated to 50° C. After 18 hours, the reaction was concentrated in vacuo and purified by flash chromatography on silica gel (CH₂Cl₂/EtOAc: 4:1 to 1:1). Zolimidine was obtained (28 mg, 0.10 mmol, 82%) as an off white solid.

m.p. 240-242° C. ¹H NMR (400 MHz, CDCl₃) δ=8.21-8.10 (3H, m), 8.06-7.93 (3H, m), 7.65 (1H, d, J=9.1Hz), 7.31-7.18 (1H, m), 6.84 (1H, t, J=6.8Hz), 3.10 (3H, s). ¹³C NMR (101 MHz, CDCl₃) δ=146.0, 143.6, 139.4, 139.3, 128.0, 126.7, 126.0, 125.7, 117.9, 113.2, 109.8, 44.7. IR (umax): 1303, 1150, 775, 748, 606 cm⁻¹. HRMS (ESI) calculated for C₁₄H₁₃N₂O₂S [M+H⁺]273.0692, found 273.0698. Δ=2.2 ppm.

Synthesis of Lamotrigine

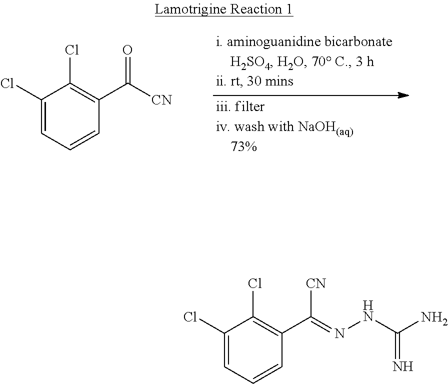 [SMILES: N#CC(=O)c1cccc(Cl)c1Cl ; N#C/C(=N\NC(=N)N)c1cccc(Cl)c1Cl]

Traditional (Glassware) Synthesis

To a stirred mixture of aminoguanidine bicarbonate (250 mg, 0.96 mmol) in sulfuric acid (4 mL 50:50 v/v with H₂O) was added 2,3-dichlorobenzoyl cyanide (250 mg, 1.27 mmol, 1.32 equiv). The reaction mixture was then heated to 70° C. for 3 hours. The reaction was then cooled to 0° C. and stirred for 30 mins. The precipitate was filtered and washed with water (2×10 mL) before being solubilized in methanol. The removal of volatiles were proceeded under vacuo to afford 2-(2,3-dichlorophenyl)-2-(guanidinylimino)acetonitrile (230 mg, 0.90 mmol, 73%) as an off-white amorphous solid.

Polypropylene Test Reaction

To a stirred mixture of aminoguanidine bicarbonate (250 mg, 0.96 mmol) in sulfuric acid (4 mL 50:50 v/v with H₂O) in a polypropylene reaction vessel was added 2,3-dichlorobenzoyl cyanide (250 mg, 1.27 mmol). The reaction mixture was then heated to 70° C. for 5 hours. The reaction was then cooled to 0° C. and stirred for 30 mins. The precipitate was filtered and washed with water (2×10 mL) before being solubilised in methanol. The removal of volatiles were proceeded under vacuo to afford 2-(2,3-dichlorophenyl)-2-(guanidinylimino)acetonitrile as (190 mg, 0.74 mmol, 59%) an off-white amorphous solid.

m.p. 175-177° C. ¹H NMR (400 MHz, DMSO-d⁶): δ=7.65 (2H, m), 7.41 (1H, dd, J=7.9Hz), 6.71 (1H, br, N—H). ¹³C NMR (101 MHz, DMSO-d⁶): δ=164.1, 135.8, 132.9, 130.5, 130.0, 129.5, 128.7, 114.9, 114.3. IR (uₘₐₓ): 3167, 3082, 3072, 3061, 1734, 1707, 1593, 1581, 1548, 1454, 1433, 1410, 1400, 1298, 1284, 1261, 1197, 1168, 1138, 1116, 1099, 1068, 1053, 1014, 976, 829, 812, 790, 744, 705, 682, 661, 628, 615 cm⁻¹. HRMS (ESI) calculated for C9H8N5Cl₂[M+H]⁺: 256.0151, found 256.0143. Δ=3.1 ppm.

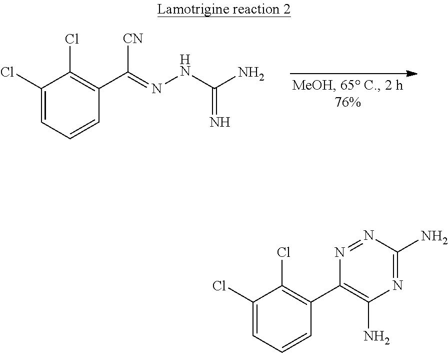 [SMILES: N#C/C(=N\NC(=N)N)c1cccc(Cl)c1Cl ; Nc1nnc(-c2cccc(Cl)c2Cl)c(N)n1]

Traditional (Glassware) Synthesis

2-(2,3-Dichlorophenyl)-2-(guanidinylimino)acetonitrile (300 mg, 1.17 mmol) was suspended in NaOH (1 M) solution and stirred for 30 mins. The solid was then filtered and washed with water (2×10 mL). The resulting solid was solubilised in methanol (6 mL) and heated to 65° C. for 2 hours after which activated carbon (20 mg), was added and the solution was stirred for a further 15 mins before being hot filtered through Celite. The resulting solution was cooled to 0° C. causing a white precipitate to form, which was filtered and washed with cold methanol to yield lamotrigine (230 mg, 0.9 mmol, 76%).

Polypropylene Test Reaction

2-(2,3-Dichlorophenyl)-2-(guanidinylimino)acetonitrile (400 mg, 1.56 mmol) was suspended in NaOH (1 m) solution and stirred for 30 mins. The solid was then filtered and washed with water (2×10 mL). The resulting solid was solubilised in methanol (6 mL) and heated to 55° C. for 6 hours. After which, activated carbon (20 mg) was added and the solution was stirred for a further 15 mins before being hot filtered through Celite. The resulting solution was cooled to 0° C. causing a white precipitate to form, which was filtered and washed with cold methanol to yield (280 mg, 1.09 mmol, 70%).

m.p. 214-216° C. ¹H NMR (400 MHz, DMSO-d⁶): δ=7.71 (1H, dd, J₁=8.0Hz, J₂=1.6Hz), 7.45(1H, dd, J₁=8.0Hz, J₂2=7.6Hz), 7.36(1H, dd, J₁=7.6Hz, J₂=1.6Hz), 6.42(2H, s). ¹³C NMR (101 MHz, DMSO-d⁶): δ 162.6, 154.6, 138.8, 137.3, 132.5, 132.1, 131.1, 128.9. IR (U_{max):} 3495, 3356, 3101, 1672, 1614, 552, 1464, 1431, 1410, 1161, 1114, 1018, 810, 788, 769, 738, 721, 605 cm⁻¹. HRMS (ESI) calculated for C₉H₇N₅Cl₂Na[M+Na]⁺: 277.9971, found 277.9927. Δ=16.8 ppm.

Modular/Monolithic Synthesis of (±)-Baclofen

Module/Monolith Preparation and Configuration

As part of the development of the Baclofen synthesis cartridge the synthesis was performed using the modules a-e (described above). First, using the modules independently to validate the procedures necessary for each stage of the synthesis, and subsequently with the modules connected with pieces of Tygon tubing to simulate the final cartridge. The inlet and outlet ports in the fluidic pathway of the reaction sequence of the modules were fitted with ¼″ UNF to either female (for inlet ports) or male (for the outlet ports) Luer-lock adaptors to allow the modules to be connected together, whilst the top inlet/outlet ports were fitted universally with ¼″ UNF to female Luer-lock adaptors. These adaptors were then fitted with either single input female to male Luer-lock connector with valve (modules a and c) or double input (female) to single output (male) Luer-lock connectors with T—valves (modules b and d). At the start of the reaction procedure all ports except the initial inlet port are sealed. Care must be taken with the order of opening/closing of individual modules to atmosphere/vacuum to avoid premature transfer of material to subsequent chambers. Starting materials and reagents were prepared and loaded into polypropylene syringes (1, 2, 5 or 20 mL capacity) either prior to beginning the synthesis or immediately prior to use. Solutions prepared in advance were stored at 4° C. prior to use in the synthesis.

Cooling and Heating Configurations

The cooling and heating of the reaction cartridge necessary for the synthesis of (±)Baclofen was achieved by immersing the lower portion of the reaction cartridge (or individual reaction module in the case of the modular implementation) in either an ice bath (cooling) or a sand bath (heating). In each case the For cooling operations the monolithic cartridge was submerged such that the lower 3 cm of the device was under the surface of the water/ice mixture to ensure the entire reaction mixture reached a sufficiently low temperature. For heating operations a sufficient quantity of sand was used such that the lower 2 cm of the device were covered, care must be taken that the coverage of the monolithic device is uniform. For some heating operations a polypropylene syringe barrel containing glass wool was attached to the appropriate chamber to act as a condenser in the case where vapors escaped the reaction vessel.

For heating and cooling operations the module was allowed to equilibrate for 15 minutes with the external environment stable at the prescribed temperature before continuing with the operations in the procedure. Heating of the sand bath was controlled by an Asynt-HP1 digital stirrer hotplate with an external temperature probe (Asynt-TC1) submerged in the sand bath. Heat flow from the sand bath to the monolithic polypropylene walls of the reactor cartridge was modelled for cartridges of varying sizes (100 mg starting material scale as designed, along with reactors at 1.25× and 1.5×) to confirm that no adjustment in the heating protocol is required based on the size of reactor for reaction scales tested in this study. Simulations were performed using SolidWorks CAD software package. Models were meshed with curvature-driven mesh (element size varying from 5 to 1 mm with 8 elements fitted per arc). The simulation was run in transient mode (900 seconds with 30 second step) with direct sparse solver. 2000 W heating was applied from the bottom plane of the sand bath. Initial temperature was set to 290 K for both sand and model. No thermal contact resistance between sand and model was defined.

Considerations

To ensure the completion of the synthesis of (±)-baclofen in the monolithic device at a desirable level, care should be taken on the following points:
During 3D printing of the monolithic device care should be taken that the printing is not paused for too long (>30 minutes) for the insertion of non-printed parts to ensure successful printing of the device.
The monolithic device should be checked for leaks to ensure the reaction sequence is not contaminated from the outside environment and that the fluidic transfers from chamber to chamber will occur smoothly.
Ensure all reagent solutions concentrations are correct. In the preferred kits of the invention, a user is provided with reagents, solvents and/or catalysts, and these may be provided at the necessary concentrations and to the necessary level of purity to allow for the production of the target product at the desired yield and purity.
Ensure that the correct solution is used for each stage in the synthesis and that the order of reagent addition is as described. The preferred kits of the invention are provided with detailed instructions that specify the order and timing of addition for each reagents, solvents and/or catalyst. In this way, the synthesis of the target product may be performed as intended, and with the reduced possibility of procedural errors.
Ensure that magnetic stirring bars have been added to all the necessary module chambers.
Once stirring is turned on, the chambers with magnetic stirring bars should be checked to ensure the bars are in motion.
When preparing the device for heating/cooling, check that the device is not in contact with the bottom of the ice/sand bath to ensure uniform temperature distribution around the device.
For heated and cooled operations, ensure sufficient time has been left to allow reaction mixtures to achieve a stable temperature before continuing with the procedure.
Care must be taken that the valve positions are adhered to in order to prevent premature transfer of material from one chamber to the next.

As noted in the comments above, the use of the reactionware, and the performance of the multistage reaction may call for careful setting up of the reactionware. The user may be provided with operating instructions

Care must be taken not to exceed the flow rates specified for extraction operations as elevated pressures can force aqueous phase through the hydrophobic frits leading to inefficient separation. Synthetic procedure.

The procedure for the modular synthesis is given below, firstly as prose and then as an itemized list of precise operations which, when followed, allow the operator to conduct the synthesis of (±)-baclofen in the monolithic implementation with minimal human interaction. We have also developed a precise digital code (using the programming language python) that can accompany the synthesis and be loaded into a digital stirrer hotplate to automatically perform the hotplate operations at the appropriate times. This code is available from the authors upon request.

The valve on the top port of the first module is opened to avoid buildup of pressure within the device as material is added, this port is connected to a slight overpressure of nitrogen supplied via Schlenk line. Solutions of methyl 4-chloro-cinammate (0.5 m, 100 mg, 0.5 mmol) and tetra butyl ammonium fluoride (TBAF) (1 m, 1 mL, 1 mmol) in THF and 0.3 mL of nitromethane (5.6 mmol) are introduced sequentially into the first reaction chamber. This reaction mixture is stirred for 3 hours. Once the reaction was complete the chamber is put under vacuum (via the aforementioned Schlenk line) for 1 hour to evaporate a significant proportion of the THF present. A 2 M aqueous solution of NH₄Cl (2 mL) is then introduced to ensure that the tetrabutyl ammonium ions remain in the aqueous phase of the subsequent ether extraction. Once this has stirred for 10 minutes, 20 mL of Et2O is flowed through the reaction chamber at a rate of 1 mL/min under stirring. The less dense etheric phase rises through the hydrophobic frit and drains into the next reaction chamber, ensuring none of the aqueous material is carried through into the next reaction chamber. Once all the ether has been pumped through the chamber any remaining liquid (aqueous phase and excess Et2O) was drawn back through the inlet opening before carrying on to the next stage of the reaction.

Once in the second chamber, (b), the extraction solvent was evaporated under reduced pressure. Once the solvent has been removed a solution of NiCl₂.6H₂O (137 mg, 0.6 mmol) in methanol (2 mL) was introduced to the chamber and the reaction chamber cooled to 0° C. A freshly prepared solution of NaBH₄ (90 mg, 2.4 mmol) in ethanol (3 mL) was then introduced to the chamber which was slowly dripped into the chamber through the top opening at a rate of 0.5 mL/min. The reduction reaction is allowed to continue for 1 hour before being brought up to room temperature and a 2 mL aqueous solution of K2CO₃ (200 mg, 1.4 mmol) was introduced to the chamber. The reaction had then stirred for 1 hour to ensure complete conversion of the material to the corresponding lactam, vacuum was again applied to the chamber along with mild heating (40° C.) for 1 hour to gently remove the alcohols present in the solvent mixture. Once the reaction mixture had reduced sufficiently in volume, further water was added to the chamber followed by CH₂Cl₂ (20 mL) at a rate of 1 mL/minute.

The openings of the chamber were arranged such that the introduction of the extraction solvent pushed the material through the fluidic channel connecting module (b) with module (c). The organic layer is transferred directly to the upper chamber and passes through the frit and flows directly into the next reaction chamber, (d). After the completion of the organic phase transfer, the solvent is removed under a flow of air. An aqueous 6 m HCl solution (3 mL) is added to the reaction chamber and the mixture heated to 100° C. for 24 hours. After cooling the acidic medium is again removed under a flow of air along with heating (70° C.), followed by the addition of MeOH (3mL) to the chamber to dissolve the reaction residue. Diethyl ether (20 mL) is added and the chamber is pressurized to transfer the slurry through to the final (filtration) cartridge The Baclofen product can be recovered as a white microcrystalline solid (56 mg, representing a 44% yield over three reaction steps based on starting material (methyl 4-chlorocinnamate). Purity of the final product was assessed by HPLC as ≥95%.

(±)-Baclofen Reaction Stage NMR Comparisons

At the modular development stage, samples of crude reaction mixture for (±)-baclofen reactions 1 and 2 were taken from the modular and monolithic reactors during the synthesis to ascertain the success of each reaction and asses—the suitability of continuation to subsequent phases of the process. This comparison was carried out by ¹H NMR.

HPLC Analysis of Cartridge Synthesized Baclofen.

Material collected from the modular and cartridge synthesis of Baclofen were analysed using a Dionex 3000 Ulitmate HPLC system comprising LPG-3400SD pump, DAD-3000 detector with a 13 μL flow cell, WPS-3000TFC analytical autosampler with fraction collector, and TCC-3000SD column thermostat, running Chromeleon 6.8. A reversed-phase 018 column (Purospher STAR RP-18 endcapped (5 pm), 100×4.6 mm).

Baclofen Synthesis Scaling Experiments

The synthesis procedure described above for (±)-baclofen was carried out using the quantities of starting materials, reagents, and solvents given in Table 3 below. The 300 mg scale reaction was carried out in a modified device in which the capacity of the initial reaction chamber was increased to 6.5 mL to accommodate extra reaction medium.

TABLE 3
Reagent quantities for different reaction scale syntheses of (±)-baclofen

| Reagent | Quantity | | |
|---|---|---|---|
| methyl 4-chloro- | 100 mg | 200 mg | 300 mg |
| cinammate | (1 mL 0.5 M | (1 mL 1.0 M | (1.5 mL 1.0 M |
| | in THF) | in THF) | in THF) |
| TBAF (1 M in THF) | 1 mL | 2 mL | 3 mL |
| MeNO₂ | 0.3 mL | 0.6 mL | 0.9 mL |
| 2 M NH₄Cl_{(aq)} | 2 mL | 3 mL | 3 mL |
| Et₂O (1^{st} extraction) | 20 mL | 25 mL | 30 mL |
| NiCl₂ · 6H₂O | 137 mg | 274 mg | 411 mg |
| NaBH₄ | 90 mg | 180 mg | 270 mg |
| MeOH (NiCl₂ · 6H₂O | 2 mL | 3 mL | 4 mL |
| solvent) | | | |
| EtOH (NaBH₄ | 3 mL | 6 mL | 9 mL |
| solvent) | | | |
| K₂CO₃ | 200 mg | 400 mg | 600 mg |
| H₂O | 2 mL | 4 mL | 6 mL |
| CH₂Cl₂ | 20 mL | 25 mL | 30 mL |
| 6 M HCl | 3 mL | 4 mL | 4 mL |
| MeOH (final product | 3 mL | 4 mL | 6 mL |
| solvent) | | | |
| Et₂O (crystallization | 20 mL | 20 mL | 20 mL |
| antisolvent) | | | |
| (±)-baclofen | 56 mg | 98 mg | 133 mg |
| hydrochloride | | | |

Single Module Synthesis of Lamotrigine.

Lamotrigine synthesis was carried out in a single filtration module in which each of the operations was carried out as follows. During fabrication of the module solid reagents aminoguanidine bicarbonate (250 mg, 0.96 mmol) and 2,3-dichlorobenzoyl cyanide (250 mg, 1.27 mmol) along with a PTFE coated magnetic stirring bar were added subsequent to the addition of the fritted glass filter. Once the module had finished printing all outlets were fitted with ¼ ″ UNF threaded Luer lock adaptors and the cartridge sealed. To initiate the reaction the input and roof adaptors were unsealed and a solution of H₂SO₄ (4 mL 50:50 v/v with H₂O) was introduced into the module at a rate of 0.5 mL/min under stirring. The reaction module was then heated to 70° C. for 3 hours. Once the reactor had cooled to room temperature the reaction mixture was washed with 20 mL H₂O and 20 mL 2 M NaOH solution, followed by a further 20 mL H₂O, with washings being removed via the drain at the bottom of the filtration cartridge. The valve on the module drain was then closed and 5 mL of MeOH introduced into the module. The module was then fitted with a condenser similar to that shown in the baclofen synthesis above and the module heated to 55° C. for 6 hours. The reactor was then cooled to 0° C. under continuous stirring before the valve on the module drain was again opened and the methanolic solution removed. The solid material inside the module was then washed with further cold methanol to yield lamotrigine as a pale yellow solid (112 mg, 0.44 mmol, 46%).

### REFERENCES

All documents mentioned in this specification are incorporated herein by reference in their entirety.

Addolorato et al. Alcohol Alcoholism 37, 504 (2002)

Almirante et al. J. Med. Chem. 8, 305 (1965)

Camps et al. Tetrahedron: Asymmetry 15, 2039 (2004)

Crowl and Louvar, Chemical process safety: fundamentals with applications. (Pearson Education, 2001)

Da Prada et al. Life Sci. 19, 1253 (1976)

Dragone et al., Beilstein J. Org. Chem., 9, 951 (2013)

Fitton et al. Drugs 50, 691 (1995)

Kitson et al. Angew. Chem. Int. Edit 53, 12723 (2014)

Kitson et al. Beilstein J. Org. Chem. 12, 2776 (2016)

Kitson et al. Chem. Sci. 4, 3099 (2013)

Kitson et al. Nat. Protocols 11, 920 (2016)

Mann et al. J. Med. Chem. 34, 1307 (1991)

Roberge et al. Chem. Eng. Technol. 28, 318 (2005)

Symes et al. Nature chemistry 4, 349 (2012)

US 2016/147202

U.S. Pat. No. 8,934,994

1. A method for digitising a method of chemical synthesis, the method comprising the steps of:
(i) identifying a method of chemical synthesis for a target product, which may be a multistep method of synthesis;
(ii) establishing a process sequence for the method of synthesis, which process sequence is a collection of chemical and/or physical steps within the method of synthesis;
(iii) translating the process sequence to a digital model of the method of synthesis, which digital model comprises a digital description of the chemical and/or physical steps within the method of synthesis.

2. The method of claim 1, wherein the method of synthesis identified in step (i) is for a target product is a multistep method of chemical synthesis, such as having three or more reaction steps in a sequence.

3. The method of claim 2, wherein the chemical steps are interspersed with one or more purification steps.

4. The method of claim 1, wherein the process sequence established in step (ii) is the collection of chemical and physical steps within the method of synthesis.

5. The method of claim 1, wherein the physical steps include one or more steps selected from the group consisting of admixture of materials for use in the reaction, heating, cooling, degassing, irradiation and saturation.

6. The method of claim 1, further comprising the step of:
(iv) designing a digital reactionware for the method of synthesis, where the digital reactionware provides a digital reaction module for each step in the process sequence, and the digital modules are digitally interconnected for the digital production of the target product.

7. The method of claim 6, wherein the digital reactionware may be obtained from common digital components from a digital reactionware library.

8. The method of claim 7, wherein the digital reactionware library holds modules having reaction chambers and modules having purification components.

9. The method of claim 8, further comprising the step of:
(v) generating a physical reactionware from the digital reactionware, where the physical reactionware has a module for each step in the process sequence.

10. The method of claim 9, wherein physical reactionware may be generated in step (v), at least in part, by 3D printing or injection moulding, such as 3D printing, according to the design in the digital reactionware.

11. The method of claim 9, wherein the physical reactionware has a security device that is a physical feature of the physical reactionware.

12. (canceled)

13. The method of claim 9, further comprising the step of:
(vi) performing a method of synthesis in the physical reactionware for the production of a target product.

14. (canceled)

15. A kit comprising a reactionware for use in a method of synthesising a target product, which reactionware is obtained or obtainable by the method of claim 9, together with one or more reagents, catalysts and/or solvents, and optionally together with a set of instructions for preparing the target product using the reactionware together with the reagents, catalysts and/or solvents.
